(19) United States
(12) Patent Application Publication
(10) Pub. No.: US 2022/0096334 A1
(43) Pub. Date: Mar. 31, 2022

(54) SOLID HAIR CONDITIONING COMPOSITION

(71) Applicant: Henkel AG & Co. KGaA, Duesseldorf (DE)

(72) Inventors: Zhengqiao DENG, Shanghai (CN); Congying LIU, Shanghai (CN)

(73) Assignee: Henkel AG & Co. KGaA, Duesseldorf (DE)

(21) Appl. No.: 17/418,158

(22) PCT Filed: Dec. 28, 2018
(86) PCT No.: PCT/CN2018/124871

§ 371 Date: Jun. 24, 2021

(51) Int. Cl. A61K 8/02; A61K 8/34; A61K 8/41; A61Q 5/12

(52) U.S. Cl. CPC A61K 8/0216; A61K 8/342; A61K 2800/805; A61K 8/416; A61Q 5/12; A61K 8/345

(57) ABSTRACT

The present disclosure provides a solid hair conditioning composition with a low water content. The solid hair conditioning composition of the present disclosure includes: (a) from about 10 wt % to about 70 wt % of a fatty alcohol material, (b) from about 5 wt % to about 50 wt % of a cationic surfactant, and (c) from about 0.5 wt % to about 9 wt % of a polyhydric alcohol and/or polyethylene glycol, wherein the water content is less than about 20 wt %. The present disclosure also provides a solid hair conditioner obtained from the solid hair conditioning composition.

### CROSS-REFERENCE TO RELATED APPLICATION

This application is a U.S. National-Stage entry under 35 U.S.C. § 371 based on International Application No. PCT/CN2018/124871, filed Dec. 28, 2018, which was published under PCT Article 21(2).

### TECHNICAL FIELD

The present disclosure relates to a solid hair conditioning composition with a low water content. In particular, the present disclosure relates to a solid hair conditioning composition that is mixed with water when used.

### BACKGROUND

With the blooming trend of fashion hair, more and more consumers tend to have their hair colored or permed more frequently than before. Hair is damaged after a chemical treatment process, such as coloring, bleaching or perming. Meanwhile, consumer's hair suffered from various kinds of damage in daily life, such as combing, blow drying, heat, UV or urban pollution. Therefore, people use conditioner or treatment to enhance hair conditioning, repair damaged hair, and provide smoothness and softness to hair. Most of hair conditioner or treatment are of lotion/cream/paste texture, filling in bottle or jar. Consumer directly apply them on hair after picking up from containers.

At the same time, with the increasing consciousness of sustainability, people turn to seeking for products which are more sustainable and more environment friendly, such as the products which contain less water.

There are more and more innovative platforms boosting in recent years to meet consumer's high requirements. Solid hair conditioner/treatment is one of such products, which is solid texture or various shaped texture by model. It is easy to carry, uses less water as compared to traditional lotion/cream/paste texture, requires less package materials, and can be used by multi-application methods. Consumer can pre-dissolve a solid hair conditioner in hot water to obtain a cream and apply it on hair, or directly apply it on wet hair after shampooing. Both application methods impart good hair care benefits, especially to damaged hair.

EP 2316414A1 discloses a hair conditioning composition including: (a) from about 10 to about 90% by mass of one or more components selected from higher alcohols, higher fatty acids, and derivatives thereof, (b) from about 5 to about 35% by mass of a cationic surfactant, and (c) a polyhydric alcohol and/or polyethylene glycol having a melting point of 155° C. or less, wherein the endothermic peak of a gel which is formed from (a) and (b) in the composition is 50° C. or more as measured by a differential scanning calorimeter (DSC) and wherein the water content is 10% by mass or less.

U.S. Pat. No. 6,831,046B2 discloses a hair conditioner composition in solid form including (i) at least 5 wt % cationic surfactant, and (ii) at least 20 wt % fatty alcohol material, wherein the composition contains less than 80 wt % water, the composition having a shape which is a stick or bar.

There is still a need to develop a solid hair conditioning composition with a low water content, which has an excellent appearance. There is still another need to develop a solid hair conditioning composition with a low water content, which has a high hardness. There is still another need to develop a solid hair conditioning composition with a low water content, which has a good emulsion structure.

### SUMMARY

In an exemplary embodiment, the present disclosure provides a solid hair conditioning composition with a low water content, which has an excellent appearance, a high hardness and/or a good emulsion structure.

In an exemplary embodiment, a solid hair conditioning composition includes

(a) from about 10 wt % to about 70 wt % of a fatty alcohol material,
(b) from about 5 wt % to about 50 wt % of a cationic surfactant, and
(c) from about 0.5 wt % to about 9 wt % of a polyhydric alcohol and/or polyethylene glycol, wherein the water content is less than about 20 wt %.

In an exemplary embodiment, the present disclosure also provides a method for preparing the solid hair conditioning composition of the present disclosure. The method includes:
mixing all the components with sufficient heating and agitation to ensure all the components are melted and/or uniformly dispersed,
pouring the resultant liquid into a mold,
cooling the liquid into a solid, and
removing the solid from the mold.

In an exemplary embodiment, the present disclosure further provides a solid hair conditioner obtained from the solid hair conditioning composition of the present disclosure

Furthermore, in an exemplary embodiment, the present disclosure provides a method for using the solid hair conditioning composition of the present disclosure or the solid hair conditioner of the present disclosure. The method includes mixing the solid hair conditioning composition or the solid hair conditioner with water.

In an exemplary embodiment, the solid hair conditioning composition according to the present disclosure has an excellent appearance, a high hardness and/or a good emulsion structure.

### BRIEF DESCRIPTION OF THE DRAWINGS

The present disclosure will hereinafter be described in conjunction with the following drawing figures, wherein like numerals denote like elements, and:

FIG. 1 shows microphotographs of the emulsion structure of the cream of Comparative Example 1.

FIG. 2 shows microphotographs of the emulsion structure of the cream of exemplary Example 1.

FIG. 3 shows microphotographs of the emulsion structure of the cream of exemplary Example 2.

FIG. 4 shows microphotographs of the emulsion structure of the cream of Comparative Example 3.

FIG. 5 shows microphotographs of the emulsion structure of the cream of exemplary Example 3.

FIG. 6 shows microphotographs of the emulsion structure of the cream of exemplary Example 4.

FIG. 7 shows photos of the products of Comparative Examples 4-5 and exemplary Examples 5-8.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It is to be understood by one of ordinary skill in the art that the present discussion is a description of exemplary embodiments only, and is not intended as limiting the broader aspects of the present disclosure. Each aspect so described may be combined with any other aspect or aspects unless clearly indicated to the contrary. In particular, any feature indicated as being preferred or advantageous may be combined with any other feature or features indicated as being preferred or advantageous.

Unless specified otherwise, all wt % values quoted herein are percentages by weight based on total weight of the solid hair conditioning composition.

Unless specified otherwise, in the context of the present disclosure, the terms used are to be construed in accordance with the following definitions.

All the molecular weights cited therein are number-average molecular weights. The number-average molecular weight is calculated by end-group analysis.

Unless specified otherwise, as used herein, the singular forms “a”, “an” and “the” include both singular and plural referents.

The terms “comprising” and “comprises” as used herein are synonymous with “including”, “includes” or “containing”, “contains”, and are inclusive or open-ended and do not exclude additional, non-recited members, elements or process steps.

The recitation of numerical end points includes all numbers and fractions subsumed within the respective ranges, as well as the recited end points.

All references cited in the present specification are hereby incorporated by reference in their entirety.

Unless otherwise defined, all terms used in the disclosing the present disclosure including technical and scientific terms, have the meaning as commonly understood by one of the ordinary skill in the art to which this present disclosure belongs. By employing further guidance, term definitions are included to better appreciate the teaching of the present disclosure.

Solid hair conditioning composition of the present disclosure are formulated as conditioners for the treatment of hair (typically after shampooing) and subsequent rinsing.

According to the present disclosure, the solid hair conditioning composition includes

(a) from about 10 wt % to about 70 wt % of a fatty alcohol material,
(b) from about 5 wt % to about 50 wt % of a cationic surfactant, and
(c) from about 0.5 wt % to about 9 wt % of a polyhydric alcohol and/or polyethylene glycol, wherein the water content is less than about 20 wt %.

### (a) Fatty Alcohol Material

The solid hair conditioning composition of the present disclosure includes a fatty alcohol material. By “fatty alcohol material” is meant a fatty alcohol, an alkoxylated fatty alcohol, or a mixture thereof.

According to the present disclosure, fatty alcohols preferably include 12 to 22 carbon atoms, more preferably 16 to 22 carbon atoms. Examples of suitable fatty alcohols include C12-13 alcohols, C12-15 alcohols, C12-16 alcohols, C14-22 alcohols, C16-17 isoalcohols, C20-22 alcohols, arachidyl alcohol, cetyl alcohol, cetearyl alcohol, myristyl alcohol, behenyl alcohol, hexyldecycloctadecanol, tetradecyleicosanol, coconut alcohol, isocetyl alcohol, isostearyl alcohol, stearyl alcohol and lauryl alcohol. Preferably, the fatty alcohol may be selected from the group including arachidyl alcohol, cetyl alcohol, cetearyl alcohol, myristyl alcohol, behenyl alcohol, hexyldecycloctadecanol, tetradecyleicosanol, coconut alcohol, isocetyl alcohol, isostearyl alcohol, stearyl alcohol and lauryl alcohol. More preferably, the fatty alcohol may be cetearyl alcohol.

Alkoxylated (e.g. ethoxylated or propoxylated) fatty alcohols having from about 12 to about 22 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof.

The fatty alcohol material is present in an amount of about 10 wt % to about 70 wt %, preferably from about 10 wt % to about 50 wt % and more preferably from about 40 wt % to about 50 wt %, based on the total weight of the solid hair conditioning composition. If the content of the fatty alcohol is less than about 10 wt %, the final product will be difficult to remove from the mold, its shape will be changed easily by external force, and its hardness will be low, which is not conducive to storage and transportation. If the content of the fatty alcohol is more than about 70 wt %, it will be difficult for the final product to dissolve in water, and the final product will be inconvenient for usage.

### (b) Cationic Surfactant

The solid hair conditioning composition of the present disclosure include one or more cationic surfactants which are cosmetically acceptable and suitable for topical application to the hair.

Preferably, the cationic surfactant according to the present disclosure can be selected from the group including Distearoylethyl Hydroxyethylmonium Methosulfate, Soytrimonium Chloride, Octacosatrimonium Chloride, Ceteartrimonium Chloride, Tallowtrimonium Chloride, Dodecylhexadecyltrimonium Chloride, Octyldodecyltrimonium Chloride, Cocotrimonium Chloride, Steartrimonium Chloride, Laurtrimonium Chloride, Cetrimonium Chloride, Dodecylbenzyltrimonium Chloride, Di-C12-15 Alkyl Dimonium Chloride, C12-18 Dialkyldimonium Chloride, Di-C12-18 Alkyl Dimonium Chloride, Dicapryl/Dicaprylyl Dimonium Chloride, Didecyldimonium Chloride, Dicetyldimonium Chloride, Ditallowdimonium Chloride, Dicocodimonium Chloride, Distearyldimonium Chloride, Ceteardimonium Chloride, Behenalkonium Chloride, Olealkonium Chloride, Hydroxyethyl Oleyl Dimonium Chloride, Cinnamidopropyltrimonium Chloride, Lactamidopropyl Trimonium Chloride, Behenoyl Pg-Trimonium Chloride, Stearoxypropyltrimonium Chloride, Lauroyl Pg-Trimonium Chloride, Palmitamidopropyltrimonium Chloride, Ricinoleamidopropyltrimonium Chloride, Panthenyl Hydroxypropyl Steardimonium Chloride, Hydroxypropyl Bis-Hydroxyethyldimonium Chloride, Hydroxypropyl Bisstearyldimonium Chloride, Hydroxycetyl Hydroxyethyl Dimonium Chloride, Hydroxyethyl Behenamidopropyl Dimonium Chloride, Behenamidopropyl Pg-Dimonium Chloride, Cocamidopropyl Pg-Dimonium Chloride, Oleamidopropyl Pg-Dimonium Chloride, Behentrimonium Chloride and any combinations thereof. More preferably, the cationic surfactant according to the present disclosure can be selected from the group including Distearoylethyl Hydroxyethylmonium Methosulfate, Behentrimonium Chloride and the combination thereof. Most preferably, the cationic surfactant according to the present disclosure may be Distearoylethyl Hydroxyethylmonium Methosulfate and Behentrimonium Chloride. In a preferable embodiment, the weight ratio of Distearoylethyl Hydroxyethylmonium Methosulfate to Behentrimonium Chloride in the solid hair conditioning composition of the present disclosure is suitably from about 10:1 to about 1:10, preferably from about 9:1 to about 1:4, more preferably from about 8:1 to about 1:2.

The cationic surfactant is present in an amount of about 5 wt % to about 50 wt %, and preferably from about 5 wt % to about 20 wt %, based on the total weight of the solid hair conditioning composition. If the content of the cationic surfactant is less than about 5 wt %, the final product will be difficult to dissolve in water and inconvenient for usage. If the content of the cationic surfactant is more than about 50 wt %, the final product will be irritating to skin and scalp, and will have a low conditioning performance.

(c) Polyhydric Alcohol and/or Polyethylene Glycol

According to the present disclosure, the solid hair conditioning composition contains a polyhydric alcohol and/or polyethylene glycol. Preferably, the polyhydric alcohol and/or polyethylene glycol has a melting point of less than about 155° C.

Examples of the polyhydric alcohol used in the present disclosure include erythritol, maltitol, sorbitol, xylitol and glycerin.

The polyethylene glycol includes a polyethylene glycol having a molecular weight of about 300 to about 5,000, 000, and the like.

The hair conditioning composition of the present disclosure can contain the polyhydric alcohol and polyethylene glycol alone or as a combination of two or more kinds. Particularly, erythritol, maltitol, and/or a polyethylene glycol having a molecular weight of about 3,000 to about 300,000 (preferably about 3,000 to about 100,000) are preferably used, and erythritol is particularly preferably used.

The polyhydric alcohol and/or polyethylene glycol is present in an amount of about 0.5 wt % to about 9 wt %, preferably from about 1 wt % to about 8 wt % and more preferably from about 2 wt % to about 7 wt %, based on the total weight of the solid hair conditioning composition. If the content of the polyhydric alcohol and/or polyethylene glycol is less than about 0.5 wt %, the final product will have a low hardness and will be easy to deform, which is not conducive to storage and transportation. If the content of the polyhydric alcohol is more than about 9 wt %, the polyhydric alcohols will be not soluble in the finish product, and thus, the appearance of the finish product will be uneven. If the content of the polyethylene glycol is more than about 9 wt %, the optical micrograph of the final product water solution will show an uneven emulsion structure.

The weight ratio of component (a) to component (b) in the solid hair conditioning composition of the present disclosure is suitably from about 10:1 to about 1:10, preferably from about 9:1 to about 1:4, more preferably from about 8:1 to about 1:7.

The solid hair conditioning composition of the present disclosure includes less than about 20 wt % of water, preferably less than about 15 wt % of water.

Optional Components

The solid hair conditioning composition according to the present disclosure may contain any other components normally used in hair conditioning compositions. These other components may include cationic polymers, oils, amphoteric surfactants, nonionic surfactants, moisturizers, thickeners, coating agents, UV absorbers, metal ion sequestering agents, pH adjusters, skin nutrients, vitamins, antioxidants, antioxidant aids and perfumes. Each of these components will be present in an amount effective to accomplish its purpose. Generally these optional components are included individually at a level of up to about 25 wt %, preferably of up to about 20 wt %, based on the total weight of the solid hair conditioning composition.

Examples of cationic polymers include Starch Hydroxypropyltrimonium Chloride, Guar Hydroxypropyltrimonium Chloride, Locust Bean Hydroxypropyltrimonium Chloride, Caesalpinia Spinosa Hydroxypropyltrimonium Chloride, Cassia Hydroxypropyltrimonium Chloride, Dextran Hydroxypropyltrimonium Chloride, Hydroxypropyl Guar Hydroxypropyltrimonium Chloride, Hydroxypropyl Oxidized Starch Pg-Trimonium Chloride, Hydrogenated Starch Hydrolysate Hydroxypropyltrimonium Chloride, Ginseng Hydroxypropyltrimonium Chloride, Lauryl Methyl Gluceth-10 Hydroxypropyldimonium Chloride, Acrylamidopropyltrimonium Chloride/Acrylamide Copolymer, Propyltrimoniumchloride Acrylamide/Dimethylacrylamide Copolymer, Dimethylacrylamide/Ethyltrimonium Chloride Methacrylate Copolymer, Polymethacrylamidopropyltrimonium Chloride, Acrylamidopropyltrimonium Chloride/Acrylates Copolymer, Acrylamidopropyltrimonium Chloride/Acrylamide Copolymer, Pg-Hydroxyethylcellulose Cocodimonium Chloride, Pg-Hydroxyethylcellulose Stearyldimonium Chloride, Soyamidopropalkonium Chloride, Distearoylethyl Dimonium Chloride, Dipalmitoylethyl Dimonium Chloride, Polyquaternium-1, Polyquaternium-10, Polyquaternium-104, Polyquaternium-11, Polyquaternium-15, Polyquaternium-16, Polyquaternium-18, Polyquaternium-19, Polyquaternium-2, Polyquaternium-22, Polyquaternium-24, Polyquaternium-28, Polyquaternium-30, Polyquaternium-32, Polyquaternium-33, Polyquaternium-35, Polyquaternium-37, Polyquaternium-39, Polyquaternium-4, Polyquaternium-43, Polyquaternium-44, Polyquaternium-46, Polyquaternium-47, Polyquaternium-49, Polyquaternium-50, Polyquaternium-51, Polyquaternium-52, Polyquaternium-53, Polyquaternium-55, Polyquaternium-56, Polyquaternium-57, Polyquaternium-6, Polyquaternium-61, Polyquaternium-62, Polyquaternium-63, Polyquaternium-64, Polyquaternium-65, Polyquaternium-67, Polyquaternium-68, Polyquaternium-69, Polyquaternium-7, Polyquaternium-73, Polyquaternium-74, Polyquaternium-75, Polyquaternium-76, Polyquaternium-78 and Polyquaternium-80.

Examples of oils include liquid oils, solid oils, hydrocarbon oils, and silicone oils.

Examples of liquid oils include avocado oil, camellia oil, turtle oil, macadamia nut oil, corn oil, mink oil, olive oil, rapeseed oil, egg oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, paulownia oil, Japanese tung oil, jojoba oil, germ oil, and triglycerin.

Examples of solid oils include cacao butter, coconut oil, horse fat, hardened coconut oil, palm oil, beef tallow, mutton tallow, hardened beef tallow, palm kernel oil, pork tallow, beef bone tallow, Japan wax kernel oil, hardened oil, heatsfoot oil, Japan wax, and hardened castor oil.

Examples of hydrocarbon oils include liquid paraffin, ozocerite, squalene, pristane, paraffin, ceresin, squalene, petrolatum, and microcrystalline wax.

Examples of silicone oils include linear polysiloxanes (such as dimethylpolysiloxane, methylphenylpolysiloxane, and diphenylpolysiloxane); cyclic polysiloxanes (such as octamethylcyclotetrasiloxane, decamethylcyclopentasiloxane, and dodecamethylcyclohexasiloxane); silicon resin forming three-dimensional network structure; silicone rubber; various kinds of modified polysiloxane (such as amino modified polysiloxane, polyether modified polysiloxane, alkyl modified polysiloxane, polyether/alkyl co-modified polysiloxane, fluorine modified polysiloxane, polyoxyethylene/polyoxypropylene copolymer modified polysiloxane, linear amino polyether modified polysiloxane, amidoalkyl modified polysiloxane, aminoglycol modified polysiloxane, aminophenyl modified polysiloxane, carbinol modified polysiloxane, polyglycerin modified polysiloxane, and polyglycerin/alkyl co-modified polysiloxane); dimethiconol; and acrylic silicones.

Examples of lipophilic nonionic surfactants include sorbitan fatty acid esters (such as sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2-ethylhexylate, and diglycerol sorbitan tetra-2-ethylhexylate); glycerol or polyglycerol fatty acid esters (such as glycerol mono-cotton seed oil fatty acid ester, glycerol monoerucate, glycerol sesquioleate, glycerol monostearate, glycerol α, α′-oleate pyroglutamate, and glycerol monostearate malate); propylene glycol fatty acid esters (such as propylene glycol monostearate); hardened castor oil derivatives; and glycerol alkyl ethers.

Examples of hydrophilic nonionic surfactants include POE-sorbitan fatty acid esters (such as POE-sorbitan monooleate, POE-sorbitan monostearate, and POE-sorbitan tetraoleate); POE sorbitol fatty acid esters (such as POE-sorbitol monolaurate, POE-sorbitol monooleate, POE-sorbitol pentaoleate, and POE-sorbitol monostearate); POE-glycerol fatty acid esters (such as POE-monooleates, POE-glycerol monostearate, POE-glycerol monoisostearate, and POE-glycerol triisostearate); POE-fatty acid esters (such as POE-distearate, POE-monodioleate, and ethylene glycol distearate); POE-alkyl ethers (such as POE-lauryl ether, POE-oleyl ether, POE-stearyl ether, POE-behenyl ether, POE-2-octyldodecyl ether, and POE-cholestanol ether); Pluronic type surfactants (such as Pluronic); POE/POP-alkyl ethers (such as POE/POP cetyl ether, POE/POP 2-decyltetradecyl ether, POE/POP monobutyl ether, POE/POP hydrogenated lanolin, and POE/POP glycerol ether); tetra POE/tetra POP-ethylenediamine condensates (such as Tetronic); POE-castor oil or hardened castor oil derivatives (such as POE-castor oil, POE-hardened castor oil, POE-hardened castor oil monoisostearate, POE-hardened castor oil triisostearate, POE-hardened castor oil monopyroglutamate monoisostearate diester, and POE-hardened castor oil maleate); POE-beeswax lanolin derivatives (such as POE-sorbitol beeswax); alkanolamides (such as coconut oil fatty acid diethanolamide, lauric acid monoethanolamide, and fatty acid isopropanolamide); POE-propylene glycol fatty acid esters; POE-alkylamines; POE-fatty acid amides; sucrose fatty acid esters; alkylethoxydimethylamine oxides; and trioleyl phosphate.

Examples of semi-synthetic water-soluble polymers include starch polymers (such as carboxymethyl starch and methylhydroxypropyl starch); cellulose polymers (such as methyl cellulose, ethyl cellulose, methylhydroxypropyl cellulose, hydroxyethyl cellulose, sodium cellulose sulfate, dialkyldimethylammonium sulfate cellulose, hydroxypropyl cellulose, carboxymethyl cellulose, sodium carboxymethyl cellulose, crystalline cellulose, cellulose powder, hydrophobically modified compounds of these polymers (e.g., partially stearoxy modified compounds), and cation modified compounds of these polymers); alginate polymers (such as sodium alginate and propylene glycol alginate); and sodium pectate.

Examples of synthetic water-soluble polymers include vinyl polymers (such as polyvinyl alcohol, polyvinyl methyl ether, polyvinylpyrrolidone, and carboxyvinyl polymer); polyoxyethylene polymers (such as polyoxyethylene/polyoxypropylene copolymers, for example, polyethylene glycol 20,000, 40,000 or 60,000); poly(dimethyldiallylammonium halide) type cationic polymers (such as Merquat100 manufactured by Merck & Co., Inc.); dimethyldiallylammonium halide/acrylamido copolymer type cationic polymers (such as Merquat550 manufactured by Merck & Co., Inc.); acrylic polymers (such as sodium polyacrylate, polyethyl acrylate, and polyacrylamide); polyethyleneimine; cationic polymers; magnesium aluminum silicate (veegum); polyquaternium-39; polyquaternium-47; polyquaternium-74; and (propyltrimonium chloride acrylamide/dimethyl acrylamide) copolymer.

Examples of UV absorbers include benzoic acid UV absorbers (such as p-aminobenzoic acid (hereinafter abbreviated as PABA), PABA monoglycerine ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA ethyl ester); anthranilic acid UV absorbers (such as homomenthyl N-acetylanthranilate); salicylic acid UV absorbers (such as amyl salicylate, menthyl salicylate, homomenthyl salicylate, octyl salicylate, phenyl salicylate, benzyl salicylate, and p-isopropanolphenyl salicylate); cinnamic acid UV absorbers (such as octyl cinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-β-phenylcinnamate, 2-ethylhexyl α-cyano-β-phenylcinnamate, and glyceryl mono-2-ethylhexanoyl-diparamethoxy cinnamate); benzophenone UV absorbers (such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, and 4-hydroxy-3-carboxybenzophenone); 3-(4′-methylbenzylidene)-d,l-camphor and 3-benzylidene-d,l-camphor; 2-phenyl-5-methylbenzoxazol; 2,2′-hydroxy-5-methylphenylbenzotriazol; 2-(2′-hydroxy-5′-t-octylphenyl) benzotriazol; 2-(2′-hydroxy-5′-methylphenylbenzotriazol; dianisoylmethane; 4-methoxy-4′-t-butyldibenzoylmethane; 5-(3,3-dimethyl-2-norbornylidene)-3-pentane-2-one; and triazine UV absorbers (such as 2-4[(2-hydroxy-3-dodecyloxypropyl)oxy]-2-hydroxyphenyl)-4,6-bis(2,4-dimethylphenyl)-1,3, 5-triazine and 2-4[(2-hydroxy-3-tridecyloxypropyl)oxy]-2-hydroxyphenyl)-4,6-bis(2,4-dimethylphenyl)-1,3, 5-triazine).

Examples of metal ion sequestering agents include 1-hydroxyethane-1,1-diphosphonic acid, 1-hydroxyethane-1,1-diphosphonic acid 4Na salt, disodium edetate, trisodium edetate, tetrasorium edetate, sodium citrate, sodium polyphosphate, sodium metaphosphate, gluconic acid, phosphoric acid, citric acid, ascorbic acid, succinic acid, edetic acid, and trisodium hydroxyethyl ethylenediamine triacetate.

Examples of pH adjusters include buffers such as lactic acid/sodium lactate, citric acid/sodium citrate, and succinic acid/sodium succinate.

Examples of vitamins include vitamins A, B1, B2, B6, C, and E and the derivatives thereof; pantothenic acid and the derivatives thereof; and biotin.

Examples of antioxidants include tocopherols, dibutylhydroxytoluene, butylhydroxyanisole, and gallic acid esters.

Examples of other components which can be contained include antiseptic (such as ethylparaben, butylparaben, 1,2-alkane diol (having a carbon chain length of 6 to 14) and the derivatives thereof, phenoxyethanol, and methylchloroisothiazolinone); antiphlogistic (such as glycyrrhizic acid derivatives, glycyrrhetinic acid derivatives, salicylic acid derivatives, hinokitiol, zinc oxide, and allantoin); whitening agent (such as saxifrage sarmentosa extract and arbutin); various extracts (such as phellodendron bark, goldthread, lithospermum root, paeonia albiflora, swertia japonica, birch, sage, loquat, carrot, aloe, malva sylvestris (mallow), iris, Vitis vinifera (grape), coix lacryma-jobi (job's tears), luffa cylindrica, lily, saffron, cnidium officinale, ginger, hypericum perforatum, ononis spinosa, allium sativum (gerlic), capsicum frutescens, citrus unshiu peel, angelica acutiloba, and sea alga); activator agent (such as royal jelly, photosensitizers, and cholesterol derivatives); blood circulation accelerator (such as nonylic acid vanillylamide, nicotinic acid benzyl esters, nicotinic acid β-butoxy ethyl esters, capsaicin, Zingerone, Cantharides tincture, ichthammol, tannic acid, α-borneol, tocopherol nicotinate, inositol hexanicotinate, cyclandelate, cinnarizine, tolazoline, acetylcholine, verapamil, cepharanthine, and γ-orizanol); antiseborrheic agent (such as sulfur and thianthol); anti-inflammatory agent (such as tranexamic acid, thiotaurine, and hypotaurine); and aromatic alcohols (such as benzyl alcohol and benzyloxy ethanol).

The solid hair conditioning composition according to the present disclosure can be made by any appropriate method. For example, sticks/bars can be made by simply mixing the components with sufficient heating and agitation to ensure all the components are melted and/or uniformly dispersed (e.g. in the case of any insoluble components). Typically, the compositions are heated to a temperature in the range from about 70° C. to about 95° C. If any heat sensitive materials are being used, these can be added as the composition cools and mixed in. The heated/warmed solution, still in a mobile state is then poured into a stick/bar mould, which may take the form of a dispensing container, and allowed to cool and solidify.

An alternative method of manufacture of the solid hair conditioning compositions is inject molding. A particularly suitable injection molding process is described in WO 98/53039 (Unilever). For examples, the following injection molding process has been used to make conditioner bars according to the present disclosure:

1) The full formulation was prepared in a batch mixer by mixing at 80° C. This produced a homogeneous melt which was then cooled to room temperature at which point the product was a waxy solid.
2) The product was broken down into particles sufficiently small to feed, at ambient temperature, into the hopper of a Werner Pfleiderer twin screw co-rotating extruder, with a length to diameter ratio of about 28:1 and about 30 mm diameter.
3) The extruder was operated at 100 to 150 rpm.
4) The extruder barrel was heated to produce product for injection at 50, 55, 60 and 65° C.
5) The heated, extruded product was fed to a piston injection unit and injected into a bar mold.
6) After the mold was filled it was allowed cool to ambient temperature.
7) The mold was opened and the hair conditioner solid product was removed.

Cooling may be brought about by nothing more than allowing the container and contents to cool. Cooling may be assisted by blowing ambient or even refrigerated air over the molds/containers and their contents.

The hair conditioning composition of the present disclosure can be any dosage form depending on the desired product form, such as solid compositions with any size and shape, or powdered compositions such as powder, granules or flakes. Examples of the product forms of the hair conditioning composition of the present disclosure include various forms depending on use conditions and the like, such as a form in which the powdered hair conditioning composition is individually-packaged by the amount used, a form in which the powdered composition is filled in a bottle and the required amount is taken out when used, a form in which the solid (bar) composition is put in a container when used and mixed with water, and are not particularly limited as far as it does not impair the effects of the present disclosure. In addition, in the industrial production of the conventional hair conditioner, the process of mixing the hair conditioning composition of the present disclosure with water can be applied.

As described above, it is particularly preferred that the hair conditioning composition of the present disclosure is used as a hair conditioning precursor composition before being applied to hair as a hair conditioner. In other words, the hair conditioning composition of the present disclosure can be used in the same manner as the conventional hair conditioner by mixing the appropriate amount with water or the like when used. While the specific mixing rate in the present disclosure can be properly adjusted by the amounts of the essential components contained and the compounding ratio thereof, it is normally preferred that the composition of the present disclosure is mixed with water in an amount of about 3 to about 15 times by weight. The higher the temperature of mixing water is, the higher the mixing rate is, and even water at room temperature (20 to 30° C.) can sufficiently mix the composition. In addition, the lower (less than 200 ppm) the hardness of mixing water is, the higher the mixing rate is, and even water with high hardness can sufficiently mix the composition depending on a mixing method.

Also, the hair conditioning composition of the present disclosure may be used after mixing the required amount for each use on hands or heads or may be previously mixing in a container with an appropriate size in a mass and used as the conventional conditioner.

The solid hair conditioning composition according to the present disclosure has an excellent appearance, a high hardness and/or a good emulsion structure.

The present disclosure also provides a solid hair conditioner obtained from the solid hair conditioning composition of the present disclosure.

The hair conditioner in the present disclosure refers to overall cosmetics that provide conditioning effect to hair, and examples include hair rinse, hair treatment, and hair pack. The hair conditioner can include both type of being applied to hair and spread well overall when used and thereafter washed out (rinsed) with hot water, water or the like, and type of not being washed out after application.

Furthermore, the present disclosure provides a method for using the solid hair conditioning composition of the present disclosure or the solid hair conditioner of the present disclosure, including: mixing the solid hair conditioning composition or the solid hair conditioner with water.

The following examples are intended to assist one skilled in the art to better understand and practice the present disclosure. The scope of the present disclosure is not limited by the examples but is defined in the appended claims. All parts and percentages are based on weight unless otherwise stated.

### EXAMPLES

The materials as shown in Table 1 were used in the examples.

TABLE 1

| Material | Trade Name | Supplier |
|---|---|---|
| Cetearyl Alcohol | Lanette O | BASF |
| 68.5 wt % Distearoylethyl | Dehyquart F 75 T | BASF |
| Hydroxyethylmonium | | |
| Methosulfate + 31.5 | | |
| wt % Cetearyl Alcohol | | |
| 80 wt % Behentrimonium | Genamin KDMP | Clariant GmbH |
| Chloride + 17 wt % | | |
| Isopropyl Alcohol + | | |
| 3 wt % Water | | |
| Isopropyl Myristate | Isopropylmyristate | BASF |
| Cetyl Palmitate | Cutina CP | BASF |
| 40 wt % Ozokerite + | Kahlwax 6103 | Inter-Harz GmbH |
| 30 wt % Hydrogenated | | |
| Vegetable Oil + 20 wt % | | |
| Stearyl Stearate + | | |
| 10 wt % Stearic Acid | | |
| Stearamidopropyl Dimethyl- | Tego Amid S 18 | Evonik |
| amine | | |
| 80 wt % Lactic Acid + | Purac 80 FCC | Inter-Harz GmbH |
| 20 wt % water | | |
| Phenoxyethanol | Phenoxetol | Clariant GmbH |
| Methylparaben | Methyl Paraben | Ashland |
| | FCC/NF NIPA | |
| Parfum | Parfum Fuji Beauty | International |
| | AV MOD | Flavors & |
| | | Fragrances NL |
| Water | Water, Purified | Henkel AG & Co |
| | | KGaA |
| PEG-150 | Polyglycol 6000 S | Clariant GmbH |
| PEG-450 | Polyglycol 20000 S | Clariant GmbH |
| Erythritol | Meso-Erythritol | Sinopharm |
| | | Chemical Reagent |
| | | Company Ltd. |
| Maltitol | Maltitol | Sinopharm |
| | | Chemical Reagent |
| | | Company Ltd. |

### Inventive Examples 1-8 and Comparative Examples 1-5

### Inventive Example 1

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 8 g water and 0.7 g Purac 80 FCC, 7 g Polyglycol 6000 S was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Inventive Example 2

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 13 g water and 0.7 g Purac 80 FCC, 2 g Polyglycol 6000 S was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Inventive Example 3

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 8 g water and 0.7 g Purac 80 FCC, 7 g Polyglycol 20000 S was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Inventive Example 4

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 13 g water and 0.7 g Purac 80 FCC, 2 g Polyglycol 20000 S was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Inventive Example 5

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 8 g water and 0.7 g Purac 80 FCC, 7 g Meso-Erythritol was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Inventive Example 6

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 13 g water and 0.7 g Purac 80 FCC, 2 g Meso-Erythritol was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Inventive Example 7

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 8 g water and 0.7 g Purac 80 FCC, 7 g Maltitol was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Inventive Example 8

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 13 g water and 0.7 g Purac 80 FCC, 2 g Maltitol was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Comparative Example 1

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 0.7 g Purac 80 FCC, 15 g Polyglycol 6000 S was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Comparative Example 2

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. Then, 15 g water and 0.7 g Purac 80 FCC were added into the beaker, and the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Comparative Example 3

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 0.7 g Purac 80 FCC, 15 g Polyglycol 20000 S was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Comparative Example 4

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 0.7 g Purac 80 FCC, 15 g Meso-Erythritol was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Comparative Example 5

39 g Lanette O, 4 g Dehyquart F 75 T, 10 g Isopropylmyristate, 5 g Kahlwax 6103, 2 g Tego Amid S 18, 3 g Genamin KDMP, 20 g Cutina CP and 0.3 g Methyl Paraben FCC/NF NIPA were added in a beaker, and then heated to 85° C. to melt completely. After adding 0.7 g Purac 80 FCC, 15 g Maltitol was added into the beaker. Then, the mixture was heated for another 20 minutes. After that, the mixture was cooled to 50° C. under stirring, and 0.6 g Parfum and 0.4 g Phenoxetol were added. The resultant mixture was continuously stirred until viscosity increase, but still flowable; and then, it was poured into a heart-shaped silica gel mold and a round cake-shaped silica gel mold quickly. The product was placed at room temperature until the bulk become solid, and then it was removed from the mold.

### Performance Evaluation

### A. Influence of the Content of PEG-150 on the Solid Hair Conditioning Composition

To illustrate the content of PEG-150 on the solid hair conditioning composition, Inventive Examples 1-2 and Comparative Examples 1-2 were used, and the components and its amounts of their composition are shown in Table 2, respectively.

TABLE 2

| | | Comparative | | Inventive | | Inventive | | Comparative | |
|---|---|---|---|---|---|---|---|---|---|
| | | Example 1 | | Example 1 | | Example 2 | | Example 2 | |
| Component | Cetearyl Alcohol | 40.26 | g | 40.26 | g | 40.26 | g | 40.26 | g |
| (a) | | | | | | | | | |
| Component | Distearoylethyl | 2.74 | g | 2.74 | g | 2.74 | g | 2.74 | g |
| (b) | Hydroxyethylmonium | | | | | | | | |
| | Methosulfate | | | | | | | | |
| | Behentrimonium | 2.4 | g | 2.4 | g | 2.4 | g | 2.4 | g |
| | Chloride | | | | | | | | |
| Component | PEG-150 | 15 | g | 7 | g | 2 | g | 0 | g |
| (c) | Isopropyl Myristate | 10 | g | 10 | g | 10 | g | 10 | g |
| | Cetyl Palmitate | 20 | g | 20 | g | 20 | g | 20 | g |
| | Stearyl Stearate | 1 | g | 1 | g | 1 | g | 1 | g |
| | Stearamidopropyl | 2 | g | 2 | g | 2 | g | 2 | g |
| | Dimethylamine | | | | | | | | |
| | Ozokerite | 2 | g | 2 | g | 2 | g | 2 | g |
| | Hydrogenated | 1.5 | g | 1.5 | g | 1.5 | g | 1.5 | g |
| | Vegetable Oil | | | | | | | | |
| | Lactic Acid | 0.56 | g | 0.56 | g | 0.56 | g | 0.56 | g |
| | Isopropyl Alcohol | 0.51 | g | 0.51 | g | 0.51 | g | 0.51 | g |
| | Stearic Acid | 0.5 | g | 0.5 | g | 0.5 | g | 0.5 | g |
| | Phenoxyethanol | 0.4 | g | 0.4 | g | 0.4 | g | 0.4 | g |
| | Methylparaben | 0.3 | g | 0.3 | g | 0.3 | g | 0.3 | g |
| | Parfum | 0.6 | g | 0.6 | g | 0.6 | g | 0.6 | g |
| | Water | 0.23 | g | 8.23 | g | 13.23 | g | 15.23 | g |
| SUM | | 100 | g | 100 | g | 100 | g | 100 | g |

### A.1 Emulsion Structure

Each of the compositions of Inventive Examples 1-2 and Comparative Example 1 was mixed with water at a rate of 1:4, stirred in a water bath pot at 80° C. for 2 minutes, and then stirred in ambient atmosphere for 3 minutes. The mixture thus obtained was placed until its temperature reached room temperature, and a white cream was obtained. The emulsion structures of the creams were observed through a Leica microscope (Type: DFC295 (12730469), commercially available from Leica Microsystems Ltd.) at a magnification of 100 X under natural light and polarized light, respectively.

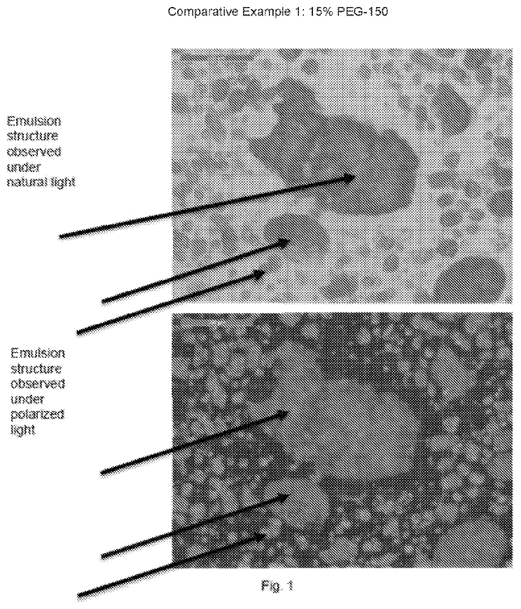

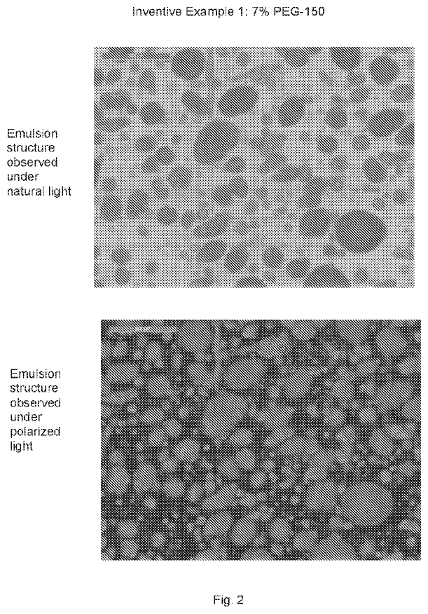

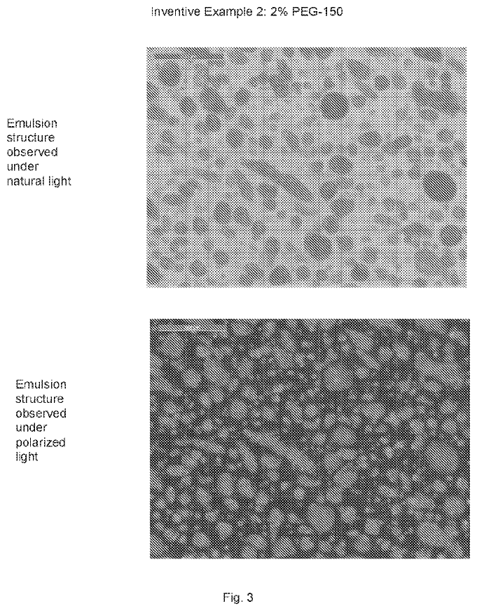

The microphotographs of the emulsion structures of the creams of Comparative Example 1 and Inventive Examples 1-2 are shown in FIGS. 1-3, respectively.

In the FIGS. 1-3, as indicated with arrows in FIG. 1, dark parts are emulsion structures of the creams. As for an emulsion structure, when the particle is smaller, and the size is more uniform, the emulsion structure is better. From FIGS. 1-3, it can be clearly seen that by comparing with the composition of Comparative Example 1, the emulsion structure of the compositions of Inventive Examples 1-2 have smaller sizes and are uniform. That is to say, as compared with the composition including 15 wt % of PEG-150 (i.e. Comparative Example 1), the compositions including 7 wt % or 2 wt % of PEG-150 according to the present disclosure (i.e. Inventive Examples 1 and 2) have better emulsion structures. Therefore, it is illustrated that the solid hair conditioning compositions including polyethylene glycol according to the present disclosure have good emulsion structures.

### A.2 Hardness

Each of the compositions of Inventive Examples 1-2 and Comparative Example 2 was made into the round cake shape, and then were measured with a Shore A Durometer (Type: Digital display LX-A, commercially available from Shanwen, Zhejiang Province, Leqing) under the same conditions, in which the samples were placed on the same position of the same table. The hardness of each sample was measured at 5 points, and the distance between different points was greater than 0.5 cm. The positions of five points taken from different samples should be similar. The test was carried out at room temperature for 5 times. The hardness values for each composition were averaged and shown in Table 3.

TABLE 3

| | Inventive | Inventive | Comparative |
|---|---|---|---|
| | Example 1 | Example 2 | Example 2 |
| Hardness value | 36.9 | 33.5 | 30.6 |
| (in HA) | | | |

As for a solid hair conditioning composition, when the hardness value is higher, the shape stability of the composition is better, and the transport and storage of the composition is more convenient. From the hardness values in Table 3, it can be seen that the compositions including 7 wt % or 2 wt % of PEG-150 according to the present disclosure (i.e. Inventive Examples 1 and 2) have higher hardness values than the composition including no PEG-150 (i.e. Comparative Example 2). Therefore, it is illustrated that the solid hair conditioning compositions including polyethylene glycol according to the present disclosure would not deform easily under external force, and the transport and storage of the compositions is convenient.

### B. Influence of the Content of PEG-450 on the Solid Hair Conditioning Composition

To illustrate the content of PEG-450 on the solid hair conditioning composition, Inventive Examples 3-4 and Comparative Examples 2-3 were used, and the components and its amounts of their composition are shown in Table 4, respectively.

TABLE 4

| | | Comparative | | Inventive | | Inventive | | Comparative | |
|---|---|---|---|---|---|---|---|---|---|
| | | Example 3 | | Example 3 | | Example 4 | | Example 2 | |
| Component | Cetearyl Alcohol | 40.26 | g | 40.26 | g | 40.26 | g | 40.26 | g |
| (a) | | | | | | | | | |
| Component | Distearoylethyl | 2.74 | g | 2.74 | g | 2.74 | g | 2.74 | g |
| (b) | Hydroxyethylmonium | | | | | | | | |
| | Methosulfate | | | | | | | | |
| | Behentrimonium | 2.4 | g | 2.4 | g | 2.4 | g | 2.4 | g |
| | Chloride | | | | | | | | |
| Component | PEG-450 | 15 | g | 7 | g | 2 | g | 0 | g |
| (c) | Isopropyl Myristate | 10 | g | 10 | g | 10 | g | 10 | g |
| | Cetyl Palmitate | 20 | g | 20 | g | 20 | g | 20 | g |
| | Stearyl Stearate | 1 | g | 1 | g | 1 | g | 1 | g |
| | Stearamidopropyl | 2 | g | 2 | g | 2 | g | 2 | g |
| | Dimethylamine | | | | | | | | |
| | Ozokerite | 2 | g | 2 | g | 2 | g | 2 | g |
| | Hydrogenated | 1.5 | g | 1.5 | g | 1.5 | g | 1.5 | g |
| | Vegetable Oil | | | | | | | | |
| | Lactic Acid | 0.56 | g | 0.56 | g | 0.56 | g | 0.56 | g |
| | Isopropyl Alcohol | 0.51 | g | 0.51 | g | 0.51 | g | 0.51 | g |
| | Stearic Acid | 0.5 | g | 0.5 | g | 0.5 | g | 0.5 | g |
| | Phenoxyethanol | 0.4 | g | 0.4 | g | 0.4 | g | 0.4 | g |
| | Methylparaben | 0.3 | g | 0.3 | g | 0.3 | g | 0.3 | g |
| | Parfum | 0.6 | g | 0.6 | g | 0.6 | g | 0.6 | g |
| | Water | 0.23 | g | 8.23 | g | 13.23 | g | 15.23 | g |
| SUM | | 100 | g | 100 | g | 100 | g | 100 | g |

### B.1 Emulsion Structure

Each of the compositions of Inventive Examples 3-4 and Comparative Example 3 was mixed with water at a rate of 1:4, stirred in a water bath pot at 80° C. for 2 minutes, and then stirred in ambient atmosphere for 3 minutes. The mixture thus obtained was placed until its temperature reached room temperature, and a white cream was obtained. The emulsion structures of the creams were observed through a Leica microscope (Type: DFC295 (12730469), commercially available from Leica Microsystems Ltd.) at a magnification of 200 X under natural light and polarized light, respectively.

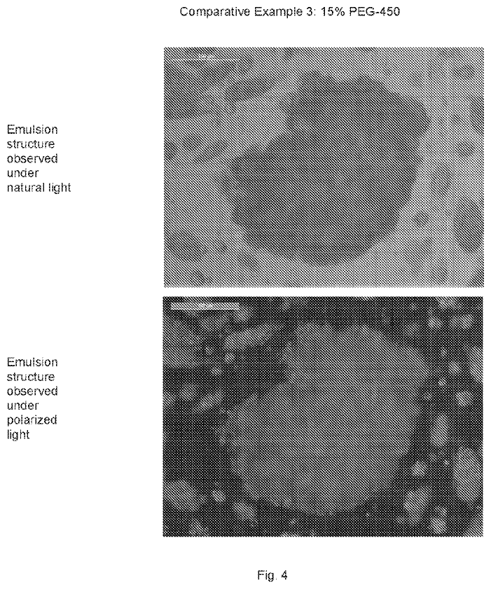

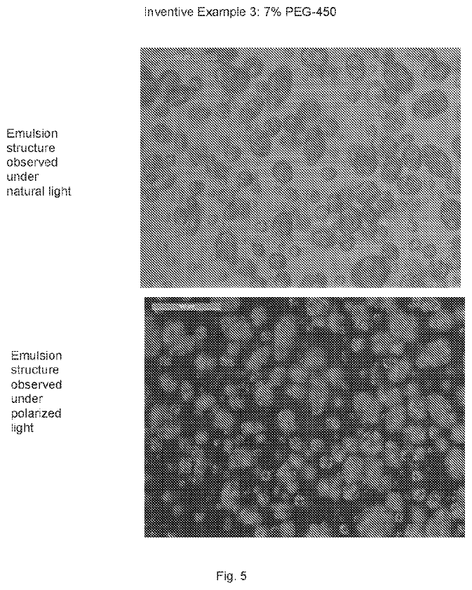

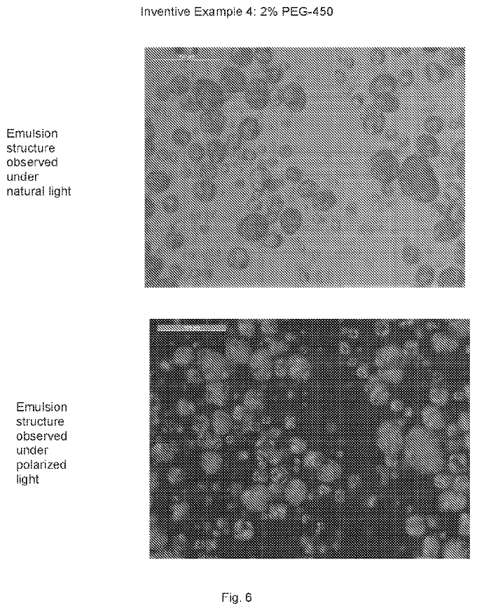

The microphotographs of the emulsion structures of the creams of Comparative Example 3 and Inventive Examples 3-4 are shown in FIGS. 4-6, respectively.

As for an emulsion structure, when the particle is smaller, and the size is more uniform, the emulsion structure is better. From FIGS. 4-6, it can be clearly seen that by comparing with the composition of Comparative Example 3, the emulsion structure of the compositions of Inventive Examples 3-4 have smaller sizes and are uniform. That is to say, as compared with the composition including 15 wt % of PEG-450 (i.e. Comparative Example 3), the compositions including 7 wt % or 2 wt % of PEG-450 according to the present disclosure (i.e. Inventive Examples 3 and 4) have better emulsion structures. Therefore, it is illustrated that the solid hair conditioning compositions including polyethylene glycol according to the present disclosure have good emulsion structures.

### B.2 Hardness

Each of the compositions of Inventive Examples 3-4 and Comparative Example 2 was made into the round cake shape, and then was measured with a Shore A Durometer (Type: Digital display LX-A, commercially available from Shanwen, Zhejiang Province, Leqing) under the same conditions, in which the samples were placed on the same position of the same table. The hardness of each sample was measured at 5 points, and the distance between different points was greater than 0.5 cm. The positions of five points taken from different samples should be similar. The test was carried out at room temperature for 5 times. The hardness values for each composition were averaged, and are shown in Table 5.

TABLE 5

| | Inventive | Inventive | Comparative |
|---|---|---|---|
| | Example 3 | Example 4 | Example 2 |
| Hardness value | 36.7 | 35.9 | 30.6 |
| (in HA) | | | |

As for a solid hair conditioning composition, when the hardness value is higher, the shape stability of the composition is better, and the transport and storage of the composition is more convenient. From the hardness values in Table 5, it can be seen that the compositions including 7 wt % or 2 wt % of PEG-450 according to the present disclosure (i.e. Inventive Examples 3 and 4) have higher hardness values than the composition including no PEG-450 (i.e. Comparative Example 2). Therefore, it is illustrated that the solid hair conditioning compositions including polyethylene glycol according to the present disclosure would not deform easily under external force, and the transport and storage of the compositions is convenient.

C. Influence of the Content of Erythritol or Maltitol on the Solid Hair Conditioning Composition

To illustrate the content of erythritol on the solid hair conditioning composition, Inventive Examples 5-6, and Comparative Examples 2 and 4 were used, and the components and its amounts of their composition are shown in Table 6, respectively.

TABLE 6

| | | Comparative | | Inventive | | Inventive | | Comparative | |
|---|---|---|---|---|---|---|---|---|---|
| | | Example 4 | | Example 5 | | Example 6 | | Example 2 | |
| Component | Cetearyl Alcohol | 40.26 | g | 40.26 | g | 40.26 | g | 40.26 | g |
| (a) | | | | | | | | | |
| Component | Distearoylethyl | 2.74 | g | 2.74 | g | 2.74 | g | 2.74 | g |
| (b) | Hydroxyethylmonium | | | | | | | | |
| | Methosulfate | | | | | | | | |
| | Behentrimonium | 2.4 | g | 2.4 | g | 2.4 | g | 2.4 | g |
| | Chloride | | | | | | | | |
| Component | Erythritol | 15 | g | 7 | g | 2 | g | 0 | g |
| (c) | Isopropyl Myristate | 10 | g | 10 | g | 10 | g | 10 | g |
| | Cetyl Palmitate | 20 | g | 20 | g | 20 | g | 20 | g |
| | Stearyl Stearate | 1 | g | 1 | g | 1 | g | 1 | g |
| | Stearamidopropyl | 2 | g | 2 | g | 2 | g | 2 | g |
| | Dimethylamine | | | | | | | | |
| | Ozokerite | 2 | g | 2 | g | 2 | g | 2 | g |
| | Hydrogenated | 1.5 | g | 1.5 | g | 1.5 | g | 1.5 | g |
| | Vegetable Oil | | | | | | | | |
| | Lactic Acid | 0.56 | g | 0.56 | g | 0.56 | g | 0.56 | g |
| | Isopropyl Alcohol | 0.51 | g | 0.51 | g | 0.51 | g | 0.51 | g |
| | Stearic Acid | 0.5 | g | 0.5 | g | 0.5 | g | 0.5 | g |
| | Phenoxyethanol | 0.4 | g | 0.4 | g | 0.4 | g | 0.4 | g |
| | Methylparaben | 0.3 | g | 0.3 | g | 0.3 | g | 0.3 | g |
| | Parfum | 0.6 | g | 0.6 | g | 0.6 | g | 0.6 | g |
| | Water | 0.23 | g | 8.23 | g | 13.23 | g | 15.23 | g |
| SUM | | 100 | g | 100 | g | 100 | g | 100 | g |

To illustrate the content of maltitol on the solid hair conditioning composition, Inventive Examples 7-8, and Comparative Examples 2 and 5 were used, and the components and its amounts of their composition are shown in Table 7, respectively.

TABLE 7

| | | Comparative | | Inventive | | Inventive | | Comparative | |
|---|---|---|---|---|---|---|---|---|---|
| | | Example 5 | | Example 7 | | Example 8 | | Example 2 | |
| Component | Cetearyl Alcohol | 40.26 | g | 40.26 | g | 40.26 | g | 40.26 | g |
| (a) | | | | | | | | | |
| Component | Distearoylethyl | 2.74 | g | 2.74 | g | 2.74 | g | 2.74 | g |
| (b) | Hydroxyethylmonium | | | | | | | | |
| | Methosulfate | | | | | | | | |
| | Behentrimonium | 2.4 | g | 2.4 | g | 2.4 | g | 2.4 | g |
| | Chloride | | | | | | | | |
| Component | Maltitol | 15 | g | 7 | g | 2 | g | 0 | g |
| (c) | Isopropyl Myristate | 10 | g | 10 | g | 10 | g | 10 | g |
| | Cetyl Palmitate | 20 | g | 20 | g | 20 | g | 20 | g |
| | Stearyl Stearate | 1 | g | 1 | g | 1 | g | 1 | g |
| | Stearamidopropyl | 2 | g | 2 | g | 2 | g | 2 | g |
| | Dimethylamine | | | | | | | | |
| | Ozokerite | 2 | g | 2 | g | 2 | g | 2 | g |
| | Hydrogenated | 1.5 | g | 1.5 | g | 1.5 | g | 1.5 | g |
| | Vegetable Oil | | | | | | | | |
| | Lactic Acid | 0.56 | g | 0.56 | g | 0.56 | g | 0.56 | g |
| | Isopropyl Alcohol | 0.51 | g | 0.51 | g | 0.51 | g | 0.51 | g |
| | Stearic Acid | 0.5 | g | 0.5 | g | 0.5 | g | 0.5 | g |
| | Phenoxyethanol | 0.4 | g | 0.4 | g | 0.4 | g | 0.4 | g |
| | Methylparaben | 0.3 | g | 0.3 | g | 0.3 | g | 0.3 | g |
| | Parfum | 0.6 | g | 0.6 | g | 0.6 | g | 0.6 | g |
| | Water | 0.23 | g | 8.23 | g | 13.23 | g | 15.23 | g |
| SUM | | 100 | g | 100 | g | 100 | g | 100 | g |

### C.1 Appearance

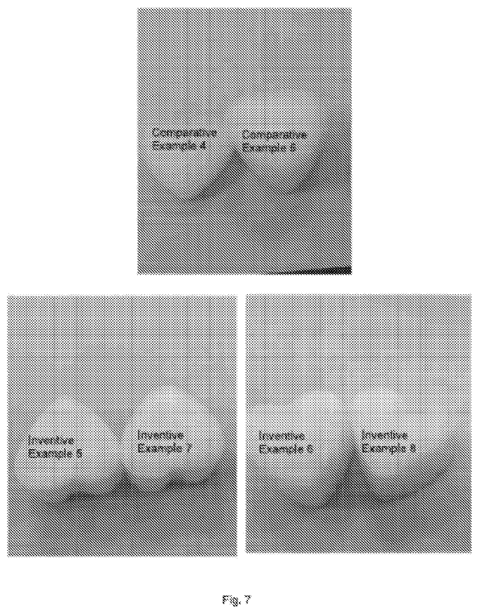

Each of the compositions of Inventive Examples 5-8 and Comparative Examples 4-5 was poured into a heart-shape mold, cooled and shaped. Photos of their products were taken by a Huawei HONOR 9 (which is commercially available from Huawei), and are shown in FIG. 7.

From FIG. 7, it can be seen that the solid products of Inventive Examples 5-8 had few particles on their surfaces and their appearance were comparatively uniform, whereas the solid products of Comparative Examples 4-5 had clearly visible particles on their surfaces. Thus, as compared with the compositions containing 15 wt % of erythritol or maltitol (i.e. Comparative Examples 4 and 5), the compositions containing 7 wt % or 2 wt % of erythritol or maltitol (i.e. Inventive Examples 5-8) have better appearances. Therefore, it is illustrated that the solid hair conditioning compositions including polyhydric alcohol according to the present disclosure have good appearances.

### C.2 Hardness

Each of the compositions of Inventive Examples 5-8 and Comparative Example 2 was made into a round cake shape, and then was measured with a Shore A Durometer (Type: Digital display LX-A, commercially available from Shanwen, Zhejiang Province, Leqing) under the same conditions, in which the samples were placed on the same position of the same table. The hardness of each sample was measured at 5 points, and the distance between different points was greater than 0.5 cm. The positions of five points taken from different samples should be similar. The test was carried out at room temperature for 5 times. The hardness values for each composition were averaged, and are shown in Table 8.

TABLE 8

| | Inventive | Inventive | Inventive | Inventive | Comparative |
|---|---|---|---|---|---|
| | Example 5 | Example 6 | Example 7 | Example 8 | Example 2 |
| Hardness value | 41.5 | 39.3 | 37.9 | 39.4 | 30.6 |
| (in HA) | | | | | |

As for a solid hair conditioning composition, when the hardness value is higher, the shape stability of the composition is better, and the transport and storage of the composition is more convenient. From the hardness values in Table 8, it can be seen that the compositions including 7 wt % or 2 wt % of Erythritol according to the present disclosure (i.e. Inventive Examples 5 and 6) and the compositions including 7 wt % or 2 wt % of Maltitol according to the present disclosure (i.e. Inventive Examples 7 and 8) have higher hardness values than the composition including no Erythritol or Maltitol (i.e. Comparative Example 2). Therefore, it is illustrated that the compositions containing polyhydric alcohol according to the present disclosure would not deform easily under external force, and the transport and storage of the compositions is convenient.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A solid hair conditioning composition, comprising
(a) from about 10 wt % to about 70 wt % of a fatty alcohol material,
(b) from about 5 wt % to about 50 wt % of a cationic surfactant, and
(c) from about 0.5 wt % to about 9 wt % of a polyhydric alcohol and/or polyethylene glycol,
wherein the water content is less than about 20 wt %.

2. The solid hair conditioning composition according to claim 1, wherein the component (a) is selected from the group of fatty alcohols having 12 to 22 carbon atoms.

3. The solid hair conditioning composition according to claim 1, wherein the content of the component (a) is from about 10 wt % to about 50 wt %, based on the total weight of the solid hair conditioning composition.

4. The solid hair conditioning composition according to claim 1, wherein the content of component (a) is from about 40 wt % to about 50 wt %, based on the total weight of the solid hair conditioning composition.

5. The solid hair conditioning composition according to any claim 1, wherein the content of the component (b) is from about 5 wt % to about 20 wt %, based on the total weight of the solid hair conditioning composition.

6. The solid hair conditioning composition according to claim 1, wherein the weight ratio of component (a) to component (b) is from about 10:1 to about 1:10.

7. The solid hair conditioning composition according to claim 1, further comprising a cationic polymer.

8. The solid hair conditioning composition according to claim 1, wherein the component (c) is a polyhydric alcohol and/or polyethylene glycol having a melting point of about 155° C. or less.

9. The solid hair conditioning composition according to claim 1 wherein the component (c) is erythritol, maltitol and/or polyethylene glycol with a molecular weight of from about 3,000 to about 300,000.

10. The solid hair conditioning composition according to claim 1, wherein the content of the component (c) is from about 1% to about 8% by weight, based on the total weight of the solid hair conditioning composition.

11. The solid hair conditioning composition according to claim 1, wherein the solid hair conditioning composition is a precursor composition for hair conditioner.

12. The solid hair conditioning composition according to claim 1, wherein the solid hair conditioning composition is configured to be mixed with water at a rate of about 3 to about 15 times by weight before use.

13. A method for preparing the solid hair conditioning composition according to claim 1, comprising:
mixing and heating all the components that form the solid hair conditioning composition to ensure all the components are melted and/or uniformly dispersed,
pouring the resultant liquid into a mold,
cooling the liquid into a solid, and
removing the solid from the mold.

14. A solid hair conditioner obtained from the solid hair conditioning composition according to claim 1.

15. A method for using the solid hair conditioning composition according to claim 1, comprising:
mixing the solid hair conditioning composition or the solid hair conditioner with water.
